(19) United States
(12) Patent Application Publication
(10) Pub. No.: US 2025/0107920 A1
(43) Pub. Date: Apr. 3, 2025

(54) DIRECT DISPOSAL URINE COLLECTION SYSTEM AND RELATED METHODS BACKGROUND

(71) Applicant: PUREWICK CORPORATION, Covington, GA (US)

(72) Inventors: Eric Alan Fallows, Apex, NC (US); Reagan Belflower, Atlanta, GA (US); Seth Schneider, Social Circle, GA (US); Miguel Eduardo Herrejon, Juarez, Chihuahua (MX)

(21) Appl. No.: 18/834,340

(22) PCT Filed: Feb. 3, 2022
(86) PCT No.: PCT/US2022/015073

§ 371 Date: Jul. 30, 2024

(51) Int. Cl. A61F 5/455; A61F 5/44; A61F 5/453

(52) U.S. Cl. CPC A61F 5/455; A61F 5/4405; A61F 5/4408; A61F 5/453

(57) ABSTRACT

Examples relate to urine management systems and methods of use of the system. The urine management system may include a urine collection device configured to receive fluid discharged from a user, a tube in fluid communication with the urine collection device configured to drain the urine, the tube having a free end for directly drawing the urine from the urine collection device and deposited directly into a toilet. The urine management system further including a pump in fluid communication with the urine collection device and the tube. The pump is configured to draw urine from the urine collection device through the tube and into the toilet.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional fluid collection devices also may be limited to use when a patient is confined to a chair or wheelchair.

Thus, users and manufacturers of fluid collection devices continue to seek new and improved devices, systems, and methods to collect bodily fluids such as urine.

### SUMMARY

Embodiments disclosed herein are related to urine management systems and methods of using the urine management systems. In an embodiment, a urine management system is disclosed. The urine management system may include a urine collection device configured to receive fluid discharged from a user and a tube in fluid communication with the urine collection device configured to drain the urine. In some embodiments, the tube may not be attached to an external collection container such that the urine is drawn from the urine collection device and deposited directly into a toilet. The tube may have a free end for directly drawing the urine from the urine collection device directly into a toilet. The urine management system may further include a pump in fluid communication with the urine collection device and the tube. The pump may be configured to draw urine from the urine collection device through the tube and into the toilet.

In an embodiment, a urine management system may include a wearable urine capture device. The urine management system may also include a first tube having a first end connected to the urine capture device and a second end connected to a suction of a pump. The urine management system may also include a second tube having a first end connected to a discharge of the pump and a second end disposed in a toilet.

In an embodiment, a method of evacuating urine by a user is disclosed. The method may include positioning a urine collection device at least proximate to a urethra of a user. The method may also include connecting a tube to the urine collection device. The method may also include drawing urine from the urine collection device into the tube with a pump and discharging the urine directly into a toilet and de-energizing the pump and disconnecting the tubing from the urine collection device. In some embodiments, positioning a urine collection device at least proximate to a urethra of a user may include integrating the urine collection device into a garment of the user. In other embodiments, positioning a urine collection device at least proximate to a urethra of a user may include removably attaching the urine collection device to a garment of the user.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a urine management system, according to an embodiment.

FIG. 1B is an isometric view of a urine management system, according to an embodiment.

FIG. 2A is an isometric view of a urine collection device, according to an embodiment.

FIG. 2B is a cross-sectional view of the urine collection device of FIG. 2A.

FIG. 3A is an isometric view of a urine collection device, according to an embodiment.

FIG. 3B is a cross-sectional view of the urine collection device of FIG. 3A.

FIG. 4A is a front view of a garment for fluid collection, according to an embodiment.

FIG. 4B is a side view schematic of the garment of FIG. 4A, according to an embodiment.

FIG. 4C is a side view schematic of the garment of FIG. 4A, according to an embodiment.

FIG. 4D is an isometric view of the back of the garment of FIG. 4A, according to an embodiment.

FIG. 4E is a front view of a garment for fluid collection, according to an embodiment.

FIG. 5A is a cross-sectional view of a pump, according to an embodiment.

FIG. 5B is a cross-sectional view of a pump, according to an embodiment.

FIG. 5C is an isometric view of a pump connected to a wheel chair according to an embodiment.

FIG. 5D is a side view schematic of a pump integrated into a garment for fluid collection, according to an embodiment.

FIG. 6 is a flow diagram of a method for evacuating urine, according to an embodiment.

### DETAILED DESCRIPTION

Embodiments disclosed herein are related to urine management systems and methods of using the same. The devices and systems disclosed herein are configured to collect fluids from an individual. The fluids collected by the fluid collection devices may include at least one of urine, vaginal discharge, penile discharge, reproductive fluids, blood, sweat, or other bodily fluids. Embodiments disclosed herein are related to chair or wheelchair mountable urine management systems and related methods. Many users of fluid collection devices are over 65 years old with limited mobility, often relying on wheelchairs as a primary mode of transportation. Many users also spend a significant amount of their day in a seated. Users and caregivers, then, are benefited from a urine management system that may be both discrete and mobile, allowing users to use the urine management system to collect fluid both at home and on the go.

In many embodiments described herein, a urine management system is configured to be used by a person that may use a wheelchair. Embodiments of the urine management system described herein may allow the user to void urine without having to transfer from the wheelchair to a toilet, which can be dangerous. In some embodiments, the urine management system also doesn't rely on a collection container, which requires emptying and cleaning, which can be tedious and disagreeable to the user and/or a caregiver.

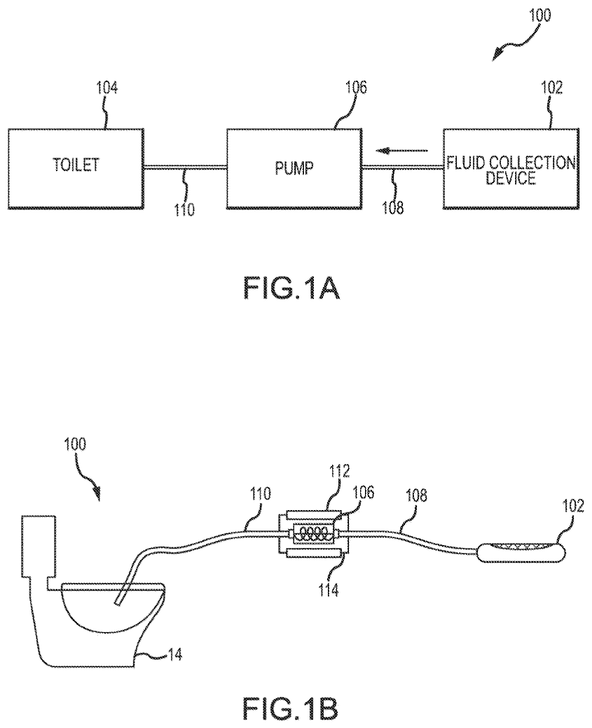

FIG. 1A is a block diagram of a urine management system 100, according to an embodiment. The urine management system 100 may be included in embodiments of urine management systems described herein. The system 100 includes a fluid (e.g., urine) collection device 102 (e.g., any of the urine collection assemblies disclosed herein), a toilet or other waste container 104, and a pump 106. The urine collection device 102 and the pump 106 may be fluidly coupled to each other via one or more tubes. For example, urine collection device 102 may be in fluid communication to the pump 106 via a first tube 108 and the pump 106 may be in fluid communication with the toilet 104 via a second tube 110. In some embodiments, the pump 106 may be configured to receive urine from the urine collection device 102, the urine may be drawn into and temporarily contained within the pump 106, and then the pump 106 may discharge the urine to the toilet 104 without the first tube 108 being attached to an external collection container such that the urine is drawn from the urine collection device 102 directly into the toilet 104. In other embodiments, the pump 106 may be disposed within or directly attached to the urine collection container 104. Fluid (e.g., urine or other bodily fluids) collected in the urine collection device 102 may be removed from the urine collection device 102 via the first tube 108 coupled to the urine collection device 102. Suction force may be introduced into the urine collection device 102 via the first tube 108 responsive to suction (e.g., vacuum) force applied at the pump 106.

FIG. 1B is an isometric view of urine management system 100, according to an embodiment. The toilet 104 may also include any other suitable waste container. The pump 106 can include a motor 112 operably connected to the pump 106. The pump 106 may be configured to draw urine from the urine collection device 102 into the toilet 104 solely responsive to the operation of the pump 106. The pump 106 may include one or more of a manual vacuum pump, syringe, a centrifugal pump, a displacement pump, a magnetically driven pump, a peristaltic pump, or any pump configured to produce a vacuum or any suitable pump configured to produce a vacuum. The pump 106 may provide a vacuum or suction to remove fluid from the urine collection device 102. In some embodiments, the pump 106 may include a screw-type positive displacement pump. Some embodiments of the pump 106 are described in more detail below.

In some examples, the motor 112 may be powered by one or more of a power cord (e.g., connected to a power socket), one or more batteries 114, or an automatic controller or a remote operator. In some examples, the motor 112 may be sized and shaped to fit around or coupled to the pump 106. In some embodiments, the pump 106 may be configured to couple outside of, on, or within the urine collection device 102. The pump 106 may include one or mechanical devices operating in combination to draw a vacuum. In some embodiments, the motor 112 may include an electric motor.

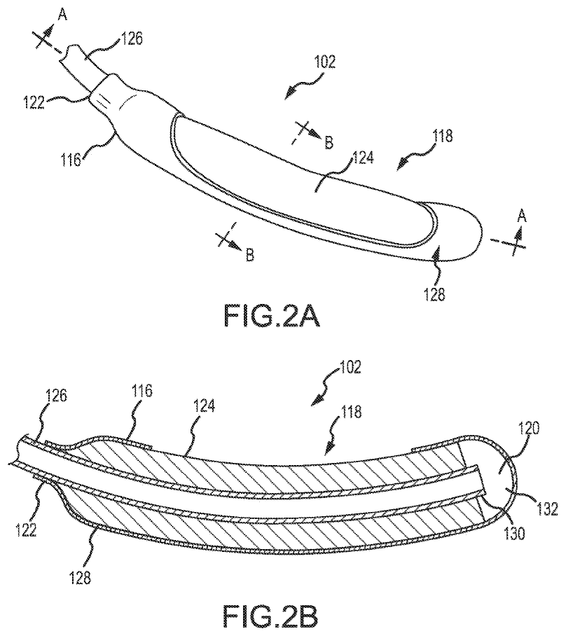

The urine management system 100 may include the urine collection device 102. The urine collection device 102 may include a male or female fluid collection device configured to be positioned at least proximate to a urethra to receive fluid discharged from a user. PCT International Application No. PCT/US2019/029616 describes various embodiments of both male and female urine collection devices, the disclosure of which is incorporated in its entirety by this reference. Moreover, the urine collection device 102 may be interchangeable in the urine management system 100 between different types, varieties, and sizes of male or female urine collection devices. The urine collection assemblies shown in FIGS. 1B-2B are examples of female urine collection assemblies that are configured to collect fluid(s) from females (e.g., collect urine from a female urethra). Further examples of female fluid collection assemblies are disclosed in U.S. Pat. No. 10,390,989 issued on Aug. 27, 2019, the disclosure of which is incorporated herein, in its entirety, by this reference. However, the urine collection assemblies, devices, systems, and methods disclosed herein may include male fluid collection assemblies and/or devices shaped, sized, and otherwise configured to collect fluid(s) from males (e.g., collect urine from a male urethra). Examples of male fluid collection assemblies are disclosed in U.S. Provisional Patent Applications No. 63/067,542 filed on Aug. 19, 2020 and U.S. Patent Application No. 16/433,773 filed on Jun. 6, 2019, the disclosure of which are incorporated herein, in its entirety, by this reference. Generally, the urine collection device 102 may include a surface sized to be positioned proximate or adjacent to the urethra and configured to wick fluid or other fluids away from the user. Urine or other fluids may be wicked from the surface to a reservoir in the urine collection device 102.

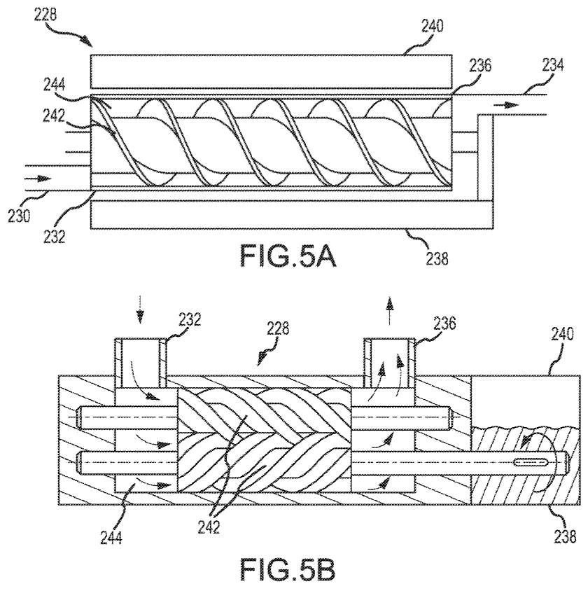

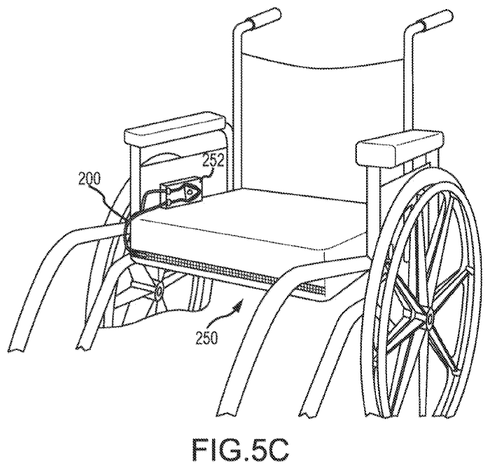

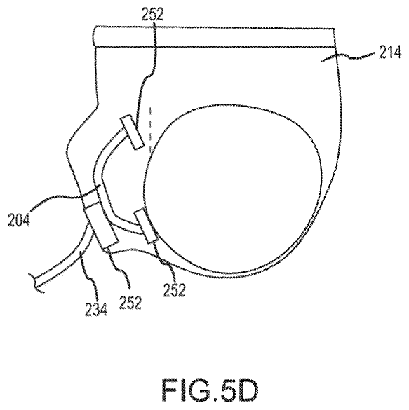

The urine management system 100 also includes a tube 108 in fluid communication with an interior region (e.g. reservoir) of the urine collection device 102 and an interior region of the suction device 106. In some embodiments, as previously discussed, the fluid collection system 100 also may include a second tube 110 providing fluid communication between the pump 106 and a toilet or waste container 104 without either the first tube 108 or the second tube 110 being attached to an external collection container. In some embodiments, the pump 106 may be coupled to or disposed within the urine collection device 102, and the first tube 108 may be absent from the urine management system 100 (as shown below in FIG. 5D). The first tube 108 and/or the second tube 110 may include a flexible tube. In some embodiments, at least a portion of the first tube 108 and/or the second tube 110 may be substantially opaque, thereby inhibiting viewing of the fluid within the first tube 108 and the second tube 110.

FIG. 2A is an isometric view of a urine collection device 102, according to an embodiment. FIG. 2B is cross-sectional view of the urine collection device 102 taken along the plane A-A of FIG. 2A. The urine collection device 102 may include a fluid impermeable barrier 116, at least one opening 118, a chamber 120, a fluid outlet 122, and a porous material 124 disposed in the chamber 120 within the fluid impermeable barrier 116. A tube 126 may be at least partially disposed within the chamber 120.

The inner surfaces of the fluid impermeable barrier 116 at least partially defines the chamber 120 within the urine collection device 102. The fluid impermeable barrier 116 temporarily stores the bodily fluids in the chamber 120. The fluid impermeable barrier 116 may be formed of any suitable fluid impermeable material(s), such as a fluid impermeable polymer (e.g., silicone rubber, thermoplastic polyurethane, polyolefins, polyvinyl chloride etc.), a metal film, natural latex rubber, another suitable material, or combinations thereof. As such, the fluid impermeable barrier 116 substantially prevents the bodily fluids from passing through the fluid impermeable barrier 116. In an at least one embodiment, the fluid impermeable barrier 116 may be air permeable and fluid impermeable, thus preventing leaks while allowing air flow through the chamber 120. In such an example, the fluid impermeable barrier 116 may be formed of a hydrophobic material that defines a plurality of pores. At least one or more portions of at least an outer surface 128 of the fluid impermeable barrier 116 may be formed from a soft and/or smooth material, thereby reducing chaffing.

In some examples, the fluid impermeable barrier 116 may be tubular (ignoring the opening), such as substantially cylindrical (as shown), oblong, prismatic, or flattened tubes. During use, the outer surface 128 of the fluid impermeable barrier 116 may contact the wearer. The fluid impermeable barrier 116 may be sized and shaped to fit in the gluteal cleft between the legs of a female user.

The opening 118 may provide an ingress route for fluids to enter the chamber 120. The opening 118 may be defined by the fluid impermeable barrier 116 such as by an inner edge of the fluid impermeable barrier 116. For example, the opening 118 may be formed in and extend through the fluid impermeable barrier 116, from the outer surface 128 to the inner surface, thereby enabling fluid(s) to enter the chamber 120 from outside of the urine collection device 102. The opening 118 may be an elongated hole in the fluid impermeable barrier 116. For example, the opening 118 may be defined as a cut-out in the fluid impermeable barrier 116. The opening 118 may be located and shaped to be positioned adjacent to a female urethra.

In some embodiments, bodily fluid may enter the chamber 120 of the urine collection device 102 via the opening 118. The urine collection device 102 may be configured to receive the fluid(s) into the chamber 120 via the opening 118. When in use, the opening 118 may have an elongated shape that extends from a first location below the urethral opening (e.g., at or near the anus or the vaginal opening) to a second location above the urethral opening (e.g., at or near the top of the vaginal opening or the pubic hair).

The urine collection device 102 may include at least one porous material 124 disposed in the chamber 120. The porous material 124 may cover at least a portion (e.g., all) of the opening 118. The porous material 124 may be configured to wick and/or allow flow of any fluid away from the opening 118, thereby preventing the fluid from escaping the chamber 120. The permeable properties referred to herein may be wicking, capillary action, diffusion, or other similar properties or processes, and are referred to herein as “permeable” and/or “wicking.” Such “wicking” and “permeable” properties may not include absorption of fluid into the porous material 124. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the porous material 124 (e.g., absorbency), such as less than about 30 wt. % of the dry weight of the porous material 124, less than about 20 wt. %, less than about 10 wt. %, less than about 7 wt. %, less than about 5 wt. %, less than about 3 wt. %, less than about 2 wt. %, less than about 1 wt. %, or less than about 0.5 wt. % of the dry weight of the porous material 124.

In an embodiment, the porous material 124 may include at least one absorbent or adsorbent material. The porous material 124 disposed within the chamber 120 may include any material that may wick and/or allow flow of the fluid. For example, the porous material 124 may be formed from fibers from nylon (e.g., spun nylon fibers), polyester, polyethylene, polypropylene, wool, silk, linen, cotton (e.g., cotton gauze), felt, other fabrics and porous polymers, hydrophobic foam, an open cell foam polyurethane,, a coated porous material (e.g., hydrophobic coated porous material, materials with affinity to specific substances), polymeric sintered particles from polyethylene, polypropylene, polytetrafluoroethylene (PTFE), elastomeric particles, any other suitable porous materials, or combinations thereof. For example, the porous material 124 may include a body of spun nylon fibers with an outer fabric gauze layers that wraps around the body of spun nylon fibers.

The tube 126 may be at least partially disposed in the chamber 120. The tube 126 may be used to remove fluid form the chamber 120. The tube 126 may be in fluid communication with the fluid outlet 122 of the urine collection device 102. The tube 126 may include a tube inlet 130 and a tube outlet (not shown) positioned downstream from the tube inlet 130. The tube outlet may be operably coupled to the pump 106. In some embodiments, the tube outlet may not be coupled to any portion of the urine management system 100, but may be configured to be disposed in the toilet or waste container 104. In some embodiments, the tube 126 may include a flexible material such as materials tubing (e.g., medical tubing). Such material tubing may include a thermoplastic elastomer, polyvinyl chloride, ethylene vinyl acetate, polytetrafluoroethylene, flexible metal, ceramic and composite material tubing etc. The tube 126 may include silicon or latex.

In some embodiments, the fluid impermeable barrier 116 may define an aperture (e.g. fluid outlet 122) sized appropriately to receive the tube 126. The tube 126 may be disposed in the chamber 120 via the fluid outlet 122. The fluid outlet 122 may be sized and shaped to form an at least substantially fluid tight seal against the tube 126 thereby substantially preventing the fluid(s) from escaping the chamber 126 via the fluid outlet 122. As shown in FIG. 2B, the inlet 130 of the tube 126 may extend through the fluid outlet 122 and into the chamber 120. In the illustrated embodiment, the tube 126 is at least partially disposed in the chamber 120. In other embodiments the inlet 30 of the tube 126 may be disposed aft of the chamber 120 or terminate at the end of the porous material 124. The fluid collected in the urine collection device 102 may be removed from the chamber 120 via the tube 126.

The urine collection device 102 may include a reservoir 132 disposed in the chamber 120. The reservoir 132 may be a substantially unoccupied portion of the chamber 120. The reservoir 132 may be at least partially defined between the fluid impermeable barrier 116 and one or both of the porous material 124 disposed in the chamber 120 and the tube inlet 130. The fluid that are in the chamber 120 may flow through the porous material 124 to the reservoir 132. The reservoir 132 may retain of the fluid therein. In some embodiments, the reservoir 132 may retain the fluid(s) temporarily, until the fluid in the reservoir 132 is removed into the tube 126. While depicted in the end region of FIG. 2B, the reservoir 132 may be located in any portion of the chamber 120. The reservoir 132 may be located in a portion of the chamber 120 that is designed to be located in a gravimetrically low point of the fluid collection device when the device is worn.

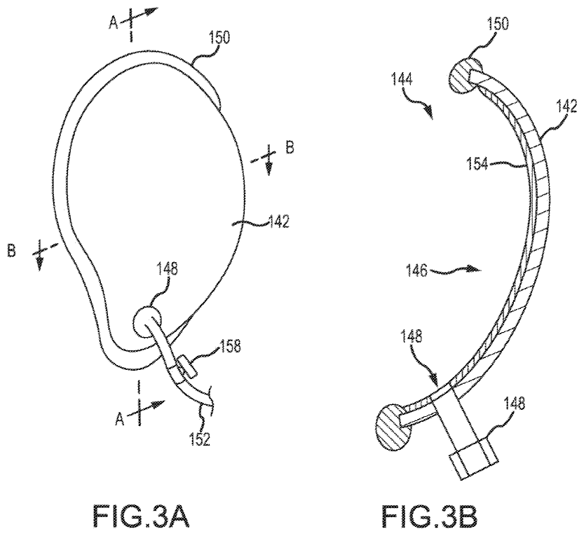

FIG. 3A is an isometric view of a urine collection device 140, according to an embodiment. FIG. 3B is cross-sectional view of the urine collection device 140 taken along the plane A-A of FIG. 3A. The urine collection device 140 may include a fluid impermeable barrier 142, at least one opening 144, a chamber 146, a connector or fluid outlet 148, and a seal 150 around a circumference of the opening 144. The inner surfaces of the fluid impermeable barrier 142 at least partially defines the chamber 146 within the urine collection device 140. The fluid impermeable barrier 142 temporarily stores the bodily fluids in the chamber 146. The fluid impermeable barrier 142 can include a connector 148 for a tube 152. In some embodiments, the fluid impermeable barrier 142 can include a concave interior surface shaped to contain the genitals of the user and the tube 150 is configured to removably couple to the fluid outlet connector 148. Similar to the embodiments discussed above, the fluid impermeable barrier 142 may be formed of any suitable fluid impermeable material(s), such as silicone or a fluid impermeable polymer (e.g., silicone rubber, thermoplastic polyurethane, polyolefins, polyvinyl chloride etc.), a metal film, natural latex rubber, another suitable material, or combinations thereof. As such, the fluid impermeable barrier 142 substantially prevents the bodily fluids from passing through the fluid impermeable barrier 142.

In some examples, the fluid impermeable barrier 142 may be cup-shaped, and/or configured to be fitted around the genitals of the user. The urine collection device 140 may include the seal 150 around the outer edge of the opening 144. During use, the seal 150 of the fluid impermeable barrier 142 may contact the wearer. The seal 150 may be formed of silicon and be sized and shaped to form a fluid tight seal around the genitals of the user (e.g. around the penis and scrotum of a male user or around the gluteal cleft between the legs of a female user).

The opening 144 may provide an ingress route for fluids to enter the chamber 146. The opening 144 may be defined by the fluid impermeable barrier 142 such as by an inner edge of the fluid impermeable barrier 144. The opening 144 may be a large hole in the fluid impermeable barrier 144, the opening 144 being an outer edge of a concave shaped fluid impermeable barrier 142. For example, the opening 144 may be defined as an outer rim of the fluid impermeable barrier 142.

In some embodiments, bodily fluid may enter the chamber 146 of the urine collection device 140 via the opening 144. When in use, the opening 144 may have a shape that extends from a first location below the urethral opening (e.g., at or near the anus or the vaginal opening) to a second location above the urethral opening (e.g., at or near the top of the vaginal opening or the pubic hair) and have a width that extends around the genitals and/or between the legs of the user. In an embodiment, portions of the chamber 146 may be substantially empty due to the varying sizes and rigidity of the male penis. Since the chamber 146 may be substantially empty (e.g., substantially all of the chamber 146 forms a reservoir), the fluids are likely to pool at a gravimetrically low point of the chamber 146. The gravimetrically low point of the chamber 146 can be a pocket formed in the cup-shaped portion of the urine collection device 140 or another suitable location (e.g., proximate to a region opposite the opening 144). The fluid outlet 148 and the inlet of the tube 152 can be positioned to be adjacent or proximate to the gravimetrically low point of the chamber 146.

The fluid impermeable barrier 144 may include at least one fluid outlet 148 that is sized and shaped to receive and seal against the tube 152, such as within the chamber 146. Accordingly, the chamber 146 may be fluidly coupled to the pump 106 via the tube 152. During operation, a male using the urine collection device 140 can discharge urine into the chamber 146. The urine and/or fluids can pool or otherwise be collected in the chamber 146, such as against the skin of the user. At least some of the fluids can enter the tube 152 via the connector 148. The fluid may be drawn out of the urine collection device 140 via the vacuum/suction provided by the pump 106. Thus, the urine may be removed through the tube 152. In some embodiments, as described above, the urine may be pumped through the pump 106 into a section of the tube 152 disposed in the toilet 104.

The urine collection device 140 may include a hydrophobic material 154 disposed in the chamber 146. Hard phobic coatings are produced with ceramic materials such as nanoaluminum, silicon dioxide, and other hard oxides, silsesquioxanes, and silanes. The hydrophobic material 154 may be spray-coated or polish-coated. The hydrophobic material 154 coating may be less than 25 μm thick and transparent, in some embodiments.

In some embodiments, the fluid outlet or connector 148 may include a valve 156. The valve 156 may include a ball valve, butterfly valve, gate valve, or other suitable valve. The valve 156 may include an operating handle that opens and closes the valve 156. In some embodiments, the user may open the valve 156 after initiating the pump 106 to draw the urine out of the urine collection device 140 in a controlled manner. In some embodiments, the valve 156 may be coupled to the tube 152 and not directly attached to the urine collection device 140 and/or the connector 148.

In some embodiments, a urine management system 200 may include a wearable urine capture device 202. The urine management system 200 may further include a first tube (e.g. tube 108) having a first end connected to the urine capture device 202 and a second end connected to a suction of a pump (e.g. pump 106). The urine management system 200 may further include a second tube (e.g. tube 110) having a first end connected to a discharge of the pump and a second end disposed in a toilet. In other words, the second end of the second tube is not attached to an external collection container such that the urine is drawn from the urine capture device 202 directly into the toilet. The pump may be configured to pull a vacuum on an interior region of the wearable urine capture device 202 effective to draw urine from the urine capture device 202 through the first tube and into the second tube.

The wearable urine capture device 202 may include a urine collection assembly 204 including a fluid impermeable barrier 206 at least defining a chamber 208, at least one opening 210, a fluid outlet 212 and a garment 214 configured to receive the urine collection assembly 204. The urine collection assembly 204 may be any of the urine collection devices described above with reference to FIGS. 2A-3B.

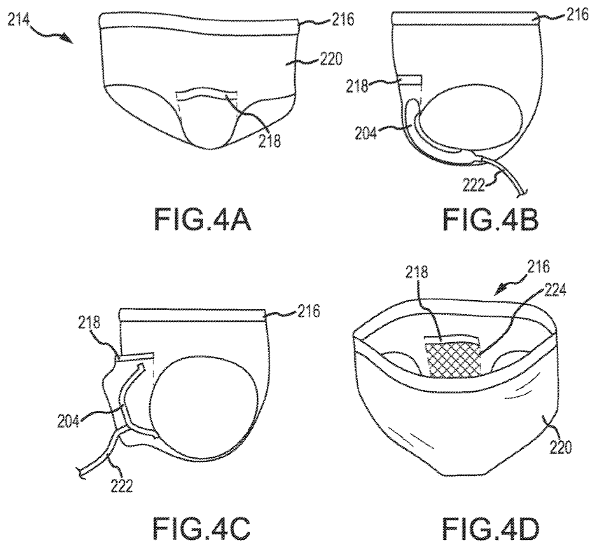

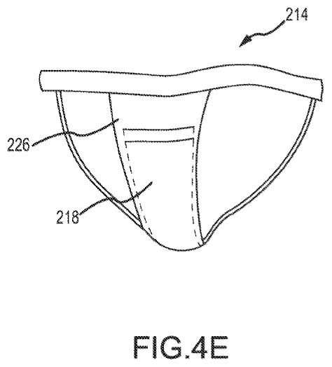

FIG. 4A is a front view of an example garment 214 for fluid collection, according to an embodiment. The garment 214 may include an undergarment having a waistband 216 that defines a waist opening and having two leg openings, the undergarment 214 having a pocket 218 disposed in the groin region configured to receive the urine collection assembly 204. The garment 214 includes a fabric body 220 that is sized and shaped to fit on a pelvis of a wearer and maintain position thereon. The fabric body 220 may be sized, shaped, and configured to form undergarments in the form of briefs. The pocket 218 may be positioned in the crotch of the fabric body 220. The pocket 218 is positioned over the region of the urethra of the wearer (e.g., within the region of the genitals) when the garment 214 is worn. A urine collection assembly (e.g., urine collection device 102 or 140) may be disposed within the pocket 218, such that fluids in the region (e.g., urine) may be collected by the urine collection assembly 204. Accordingly, the garment 214 and pocket 218 thereon may serve to align the urine collection assembly 204 with the urethra of the user such that fluids originating therefrom can be collected before soiling clothing or bedding.

FIG. 4B is a side view schematic of the garment of FIG. 4A, according to an embodiment. FIG. 4B depicts the garment 214, which includes pocket 218, which contains a female urine collection assembly 204 therein, such as urine collection device 102. The urine collection assembly 204 is sized and shaped to fit within the pocket 218. The pocket 218 is sized, positioned, and shaped to locate the urine collection assembly 204 on or over the urethra of a female wearing the garment 214. For example, the pocket 218 can be sized and positioned to receive the urine collection assembly 204 and locate the opening (not shown) of the urine collection assembly 204 on the urethral region or opening of the wearer of the garment 214. The pocket 218 may be complementary in size to the urine collection assembly 204, such as to retain the urine collection assembly 204 in a substantially fixed position within the garment 214. As shown, pocket 218 may extend from a pubic region to a perineal region. The pocket 218 may be disposed in or on the garment 214. The pocket 218 may include enough fabric to allow the urine collection assembly 204 to be inserted therein and limit movement of the urine collection assembly 204 within the pocket 218 and garment 214 when inserted. In some examples, the pocket 218 can be adjustable, such as having one or more adjustable straps to adjust the volume within the pocket 218 to retain the urine collection assembly 204.

The pocket 218 may include one or more holes therein to pass a tube 222 there through. For example, a hole may be formed in the garment 214 in a lower portion of the pocket 218 (e.g., perineal region) to allow the tube 222 to be disposed on a lowermost portion of the urine collection assembly 204 when worn. The tube 222 may be connected to the urine collection assembly 204 within the pocket 218.

FIG. 4C is a side view schematic of the garment 214 of FIG. 4A, according to an embodiment. FIG. 4C depicts the garment 214, which includes pocket 218 that contains a cup-shaped urine collection assembly therein, such as the urine collection device 140 shown in FIGS. 3A-3B. The urine collection assembly 204 is sized and shaped to fit within the pocket 218. The pocket 218 is sized, positioned, and shaped to locate the urine collection assembly 204 on or over the genitals of a user wearing the garment 214. For example, the pocket 218 can be sized and positioned to receive the urine collection assembly 204 and locate the cup portion (FIG. 3A) of the urine collection assembly over (e.g., on) the urethral region or opening of the user of the garment 214. The pocket 218 may be complementary in size to the urine collection assembly 204, such as to retain the urine collection assembly 204 in a substantially fixed position within the garment 214. As shown, pocket 218 may extend from a pubic region towards a perineal region. The pocket 218 may be disposed in or on the garment 214. The pocket 218 may include enough fabric to allow the urine collection assembly 204 to be inserted therein and limit movement of the urine collection assembly 204 within the pocket 218 and garment 214 when inserted.

The pocket 218 may include one or more holes therein to pass the tube 222 there through. For example, a hole may be formed in the garment 214 in a lower portion of the pocket 218 to allow the tube 222 to be disposed on a lowermost portion of the urine collection assembly 204 when worn. The tube 222 may be connected to the urine collection assembly 204 within the pocket 218. In some embodiments, one or more of the pocket 218 or the fabric body 220 may include a stretchable material. The stretchable material may accommodate the urine collection assembly 204 and retain the assembly within the pocket 218.

In some embodiments, the garment 214 can include a porous material to allow fluids to pass from the wearer into the urine collection assembly 204. FIG. 4D is an isometric view of the back of the garment of FIG. 4A, according to an embodiment. The garment 214 includes fabric body 220 with pocket 218 to contain the urine collection assembly 204 therein, such as urine collection device 102 or 140. As shown, pocket 218 may be located on an interior of the garment 214. FIG. 4D shows the interior of the garment 214 including the pocket 218 located in the front of the garment 214 such as in an interior pubic region of the garment 214. The pocket 218 can include a porous material 224 in a region positioned to be located between the skin of the wearer and the urine collection assembly 204. The porous material 224 may allow fluid to flow from the wearer to into the urine collection assembly within the pocket 218 with minimal absorption by the garment 214 (e.g., the material of the pocket 218). In some examples, the porous material 224 includes a hydrophobic or non-absorbent material, such as polypropylene, polyester, acrylic fibers, etc. In some examples, the porous material 224 includes a fabric mesh, such as cotton, wool, silk, rubber, polyester, acrylic fibers, any fabric disclosed herein, etc., mesh. For example, the garment 214 may include a mesh material or non-absorbent material in a position to contact the subject and the opening of the fluid collection device.

In embodiments, the fabric body 220 can be made of one or more of a fabric mesh, hydrophobic material, or non-absorbent material. In such embodiments, the pocket 218 may be made of the same material or a different material than the fabric body, such as any of the materials for a fabric body disclosed herein. Fluids emitted by the wearer can pass through the fabric body 220 and into the urine collection assembly 204 with minimal absorption by the fabric body 220.

In some examples, the garment 214 may include one or more holes or perforations in the fabric in the urethral region of the garment (e.g., region that fits over the urethra of the wearer). In such embodiments, fluid passes through the one or more holes or perforations in the fabric into the urine collection assembly. The opening of the urine collection assembly may be located at the one or more holes or perforations. In some embodiments, the garment 214 may have different forms, such as pants, shorts, briefs, boxer briefs, a thong, a jock strap, a sanitary belt, etc.

FIG. 4E is a front view of a garment 214 for fluid collection, according to an embodiment. The garment 214 may be similar or identical to the garments of FIGS. 4A-4D in one or more aspects. The garment 214 includes a strap assembly 226 that is sized and shaped to fit on a pelvis of a wearer and maintain position thereon. The strap assembly 226 may be sized, shaped, and configured to form undergarments in the form of a jock strap in some embodiments. In other words, the wearable urine capture device 202 includes a strap assembly 226 or having at least one strap attached to the urine collection assembly 204 configured to maintain the urine collection assembly 204 proximate the urethra of the user. The strap assembly 226 of the garment 214 may be similar or identical to the fabric body 220 in one or more aspects. The strap assembly 226 of the garment 214 may include pocket 218 therein. The pocket 218 of the garment 214 may be similar or identical to the pockets described in FIGS. 4A-4D in one or more aspects. For example, the pocket 218 may be positioned in the groin area of the strap assembly 226. The pocket 218 is positioned over the region of the urethra of the wearer (e.g., over the genitals) when the garment 214 is worn. A urine collection assembly 204 may be disposed within the pocket 218, such that fluids in the region (e.g., urine) may be collected by the urine collection assembly. Accordingly, the garment 214 and pocket 218 thereon may serve to align the urine collection assembly with the urethra of the user such that fluids originating therefrom can be collected before soiling clothing or bedding.

Referring now to FIGS. 5A-5B, in some embodiments, the wearable urine capture device 202 of the urine management system 200 includes a pump 228 integrated therein. Similar to the pumps described above with reference to FIGS. 1A-1B, the pump 228 may be integrated into the urine management system 200 and may be fluidly coupled to the urine capture device 202 by a first tube (e.g., tube 222). The first tube 230 may include a first end connected to the urine capture device 202 and a second end connected to a suction 232 of a pump. The urine management system 200 may include a second tube 234 having a first end connected to a discharge 236 of the pump 228 and a second end disposed in a toilet. Accordingly, a vacuum (e.g., suction) may be drawn through urine capture device 202 (e.g., urine collection device 102 or 140) via the first tube 230. As the urine is drained from the chamber of the urine capture device 202, the urine may travel through the first tube 230, through the pump 228 and pushed through the second tube 234 into a toilet or waste disposal where it may be disposed.

The pump 228 may include one or more of a manual vacuum pump, and electric vacuum pump, a diaphragm pump, a centrifugal pump, a displacement pump, a magnetically driven pump, a peristaltic pump, or any pump configured to produce a vacuum. In some examples, the pump 228 may include a motor 238 powered by one or more power sources operably coupled to the pump 228. For example, a power cord (e.g., connected to a power socket). In other examples, one or more batteries 240 may act as the power source. The pump 228 disclosed herein may include one or more of a switch, a button, a plug, a remote, or any other device suitable to activate the pump 228.

In the shown embodiment, the pump 228 includes a screw-type positive displacement pump. The screw-type positive displacement pump uses one or several screws 242 to move fluid along the screw axis in the direction of the arrows shown. In its simplest form (the Archimedes' screw pump), a single screw 242 rotates in a cylindrical cavity 244, thereby moving the urine along the screw's rotor 246. In some embodiments, the cylindrical cavity 244 can be coupled to the motor 238 and configured to rotate about the stationary screw 242, which also causes the fluid to move along the screw axis in the direction of the arrows shown. FIG. 5B illustrates a pump 228 having multiple (2) screws 242 that rotate within the cavity 244. The screws 242 may turn about the screw rotor 246 to drive the fluid through the pump 228. The pump 228 having multiple screws 242 operates using two counter-rotating screw rotors 242 which are designed to rotate “towards each other”. This traps the fluid in the space between the screws 242 of their rotors 246. As the screws rotate, this trapped volume decreases, which may compress the fluid and moves it towards the discharge 236 of the pump 228. Each screw-type pump 228 operates on the same basic principle of a screw(s) 242 turning to isolate a volume of fluid and convey it through the pump to the discharge 236. One benefit of the screw-type positive displacement pump is that the urine moves axially without turbulence which eliminates foaming. Other benefits may include noise reduction, space saving features, energy savings, cost savings, simplicity, etc. In some embodiments, the pump 228 may be disposed with the other components urine management system 200 after a single or a few uses.

Many embodiments of fluid collection systems described herein are securable or mountable to a chair or wheelchair. Turning to FIG. 5C, the urine management system 200 may be secured or detachably mounted to a wheelchair 250. The wheelchair 250 may include any of a number of different conventional wheelchairs, and may include a back, a seat and two arms. In some embodiments, the pump 252 may be mounted to an interior of the arm of the wheelchair 250. The pump 252 may be fixedly or removably secured to the wheelchair 250.

Turning to FIG. 5D, the urine management system 200 may include the urine capture device 202 that includes the urine collection assembly 204 removably attached to the garment 214. Thus, the urine collection assembly 204 may be stored outside the garment 214 (e.g., in a carrying case or pouch) and attached to the garment 214 when the user is preparing to void urine. In some embodiments, the urine collection assembly 204 may be attached to the garment 214 by at least one of a snap mechanism, Velcro, or a magnet 252.

In some embodiments, the pump 252 may be coupled directly to the urine collection assembly 204. The pump 252 may be coupled directly to a connector (e.g., connector 148) and/or mounted to the fluid impermeable barrier (e.g., fluid impermeable barrier 116 or 142). Thus, the wearable urine capture device 202 includes the pump 252 integrated therein. In some embodiments, the second tube 234 may have the first end connected to the discharge or outlet 236 of the pump 252 and the second end disposed in a toilet. Thus, in use, the urine collection assembly 204 may be attached to the garment 214 and the second end of the second tube 234 disposed in the toilet. The pump 252 may be coupled to the urine collection assembly 204 and operated to initiate the pump when before or when the user begins to urinate.

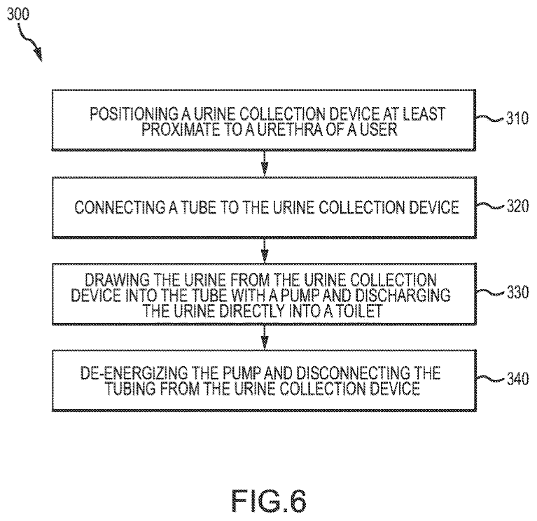

FIG. 6 is a flow diagram of a method 300 for collecting urine discharged by a user, according to an embodiment. The method 300 may include an act 310 of positioning a urine collection device at least proximate to a urethra of a user. In some embodiments, positioning a urine collection device at least proximate to a urethra of a user includes integrating the urine collection device into a garment of the user. In other embodiments, positioning a urine collection device at least proximate to a urethra of a user includes removably attaching the urine collection device to a garment of the user. The method 300 may also include an act 320 of connecting a tube to the urine collection device. The tube may be coupled to the suction of the pump. In some embodiments, a first tube may include a first end connected to the urine capture device and a second end connected to a suction of a pump. A second tube may be connected, the second tube having a first end connected to a discharge of the pump and a second end disposed in a toilet. The second end of the tube may be a free end for directly drawing the urine from the urine collection device directly into the toilet. In other words, the second end of the tube is not attached to an external collection container such that the urine is drawn from the urine collection device directly into a toilet. In some embodiments, the method 300 may include an act 330 of drawing the urine from the urine collection device into the tube with a pump and discharging the urine directly into a toilet. The method may also include an act 340 of de-energizing the pump and disconnecting the tubing from the urine collection device.

The acts of the method 300 described above are for illustrative purposes. For example, the acts of the method 300 can be performed in different orders, split into multiple acts, modified, supplemented, or combined. In an embodiment, one or more of the act of the method 300 can be omitted from the method 300. Any of the acts of the method 300 can include using any of the fluid collection systems disclosed herein.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A urine management system, comprising:
a urine collection device configured to receive fluid discharged from a user;
a tube in fluid communication with the urine collection device configured to drain the urine, wherein the tube has a free end for directly drawing the urine from the urine collection device directly into a toilet; and
a pump in fluid communication with the urine collection device and the tube, wherein the pump is configured to draw urine from the urine collection device through the tube and into the toilet.

2. The urine management system of claim 1, wherein the urine collection device comprises:
a fluid impermeable barrier at least defining a chamber, at least one opening, and a fluid outlet;
at least one porous material disposed in the chamber; and
a first end of the tube disposed in the fluid outlet and in fluid communication with the chamber.

3. The urine management system of claim 2, wherein the fluid impermeable barrier includes silicone.

4. The urine management system of claim 1, wherein the fluid collection device includes a fluid impermeable barrier at least defining a chamber and a connector for the tube, wherein the fluid impermeable barrier includes a concave interior surface shaped to contain the genitals of the user and the tube is configured to removably couple to the connector.

5. The urine management system of claim 4, wherein the concave interior surface includes a hydrophobic surface.

6. The urine management system of claim 4, wherein the connector includes a valve.

7. A urine management system, comprising:
a wearable urine capture device;
a first tube having a first end connected to the urine capture device and a second end connected to a suction of a pump;
a second tube having a first end connected to a discharge of the pump and a second end disposed in a toilet.

8. The urine management system of claim 7, wherein the wearable urine capture device includes a urine collection assembly including a fluid impermeable barrier at least defining a chamber, at least one opening, a fluid outlet, and a garment configured to receive the urine collection assembly.

9. The urine management system of claim 8, wherein the garment includes an undergarment having a waistband that defines a waist opening and having two leg openings, the undergarment having a pocket disposed in the groin region configured to receive the urine collection assembly.

10. The urine management system of either claim 8 or claim 9, wherein the wearable urine capture device includes a strap assembly having at least one strap attached to the urine collection assembly configured to maintain the urine collection assembly proximate the urethra of the user.

11. The urine management system of any of claims 8-10, wherein the urine collection assembly is removably attached to the garment by at least one of a snap mechanism, Velcro, or a magnet.

12. The urine management system of any of claims 7-11, wherein the pump is configured to pull a vacuum on an interior region of the wearable urine capture device effective to draw urine from the urine capture device through the first tube and into the second tube.

13. The urine management system of any of claims 7-12, wherein the pump is configured to detachably mount to an arm of a wheelchair.

14. The urine management system of any of claims 7-13, wherein the wearable urine capture device includes the pump integrated therein.

15. The urine management system of any of claims 7-14, wherein the system further includes a power source operably coupled to the pump.

16. The urine management system of claim 15, wherein the power source includes a battery.

17. The urine management system of any of claims 7-16, wherein the pump includes a screw-type positive displacement pump.

18. A method for evacuating urine, comprising:
positioning a urine collection device at least proximate to a urethra of a user;
connecting a tube to the urine collection device;
drawing urine from the urine collection device into the tube with a pump and discharging the urine directly into a toilet; and
de-energizing the pump and disconnecting the tubing from the urine collection device.

19. The method of claim 18, wherein positioning a urine collection device at least proximate to a urethra of a user includes integrating the urine collection device into a garment of the user.

20. The method of claim 18, wherein positioning a urine collection device at least proximate to a urethra of a user includes removably attaching the urine collection device to a garment of the user.
